(19) United States
(12) Patent Application Publication
(10) Pub. No.: US 2026/0290585 A1
(43) Pub. Date: Sep. 24, 2026

(54) MACHINE LEARNING SYSTEM FOR PREDICTING CANCER TREATMENT EFFICACY

(71) Applicant: Deep Rx Inc., Pittsburgh, PA (US)

(72) Inventors: Xinghua Lu, Pittsburgh, PA (US); Lujia Chen, Madison, CT (US); Min Sun, Cheswick, PA (US)

(73) Assignee: Deep Rx Inc., Pittsburgh, PA (US)

(21) Appl. No.: 19/379,642

(22) Filed: Nov. 4, 2025

Related U.S. Application Data
(63) Continuation-in-part of application No. PCT/US2023/068727, filed on Jun. 20, 2023.

(51) Int. Cl. G16H 50/20; G06N 20/00; G16H 20/10

(52) U.S. Cl. CPC G16H 50/20; G06N 20/00; G16H 20/10

(57) ABSTRACT

The present disclosure provides a computer-implemented system for training a machine learning model to predict efficacy of a biological agent for solid and blood cancers. The system comprises processors that execute instructions stored in computer memory. The processors identify target differentially expressed genes for a cancer using a tumor-specific causal inference algorithm applied to genomic and transcriptomic data. The processors compute expression values for metagenes from the target differentially expressed genes, wherein the metagenes exhibit co-expression patterns with respect to response to the biological agent for patients with the cancer. The processors train the model to predict the efficacy of the biological agent, such that an output is usable by a clinician for determining treatment for a new patient.

### CROSS-REFERENCE TO RELATED APPLICATIONS

This application is a continuation-in-part of U.S. application Ser. No. 18/879,529, titled MACHINE-LEARNING COMPUTER SYSTEMS AND METHODS FOR PREDICTING EFFICACY OF CHEMICAL AND BIOLOGICAL AGENTS FOR TREATING DISEASES, SUCH AS GASTROINTESTINAL CANCERS which is a National Stage Entry of PCT/US2023/068727, which claims priority to U.S. provisional patent application Ser. No. 63/355,725, filed Jun. 27, 2022, which is hereby incorporated by reference in its entirety.

### FIELD OF INVENTION

The present disclosure relates to machine learning systems for medical treatment prediction, and more particularly to a machine learning system that employs causal discovery methods to identify cancer driver genes and predict efficacy of biological and chemical agents for treating solid and blood cancers.

### BACKGROUND

Cancer treatment remains one of the most challenging areas in modern medicine, with solid and blood cancers affecting millions of patients worldwide. Traditional approaches to cancer treatment often rely on standardized protocols that may not account for the heterogeneous nature of tumors and individual patient characteristics. This one-size-fits-all approach can lead to suboptimal treatment outcomes, with some patients experiencing limited therapeutic benefit while others may suffer from adverse effects.

The complexity of cancer biology involves intricate molecular pathways and genetic alterations that drive tumor development and progression. Cancer driver genes and their associated differentially expressed genes (DEGs) play roles in cellular signaling systems that influence how tumors respond to various therapeutic interventions. Understanding these molecular mechanisms and their relationships to treatment response has become an area of active research in oncology.

Recent advances in genomic and transcriptomic profiling technologies have generated vast amounts of molecular data from cancer patients. This wealth of information presents both opportunities and challenges for clinicians seeking to make informed treatment decisions. While these datasets contain valuable insights about tumor biology and potential therapeutic targets, extracting actionable information from such complex, high-dimensional data remains a formidable task using conventional analytical approaches.

Machine learning and artificial intelligence technologies have emerged as powerful tools for analyzing complex biological datasets. These computational approaches can identify patterns and relationships within large-scale genomic data that may not be apparent through traditional statistical methods. The application of machine learning to cancer treatment prediction represents a growing field that seeks to leverage molecular information to guide therapeutic decision-making.

Current methods for predicting treatment response in cancer patients often rely on limited clinical and pathological factors. While these approaches provide some guidance, they may not capture the full spectrum of molecular factors that influence therapeutic efficacy. There exists a general need for more sophisticated computational approaches that can integrate multiple types of molecular data to provide more accurate predictions of treatment response for individual cancer patients.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a computer-implemented system for training a machine learning model to predict efficacy of a chemotherapy or a biological agent for solid and blood cancers is provided. The system comprises one or more processors that execute instructions stored in computer memory. The system computes and discovers the causal relationships between cancer-driving somatic genomic alterations (SGAs) and their target differentially expressed genes (DEGs). The system then searches for a set of metagenes among DEGs, where a metagene represents co-expression pattern and value of a set of DEGs affected by SGAs perturbing a common pathway. Finally, the system using expression values of metagenes as input features to train machine learning models to predict tumor cell responses to chemotherapy or biological drugs. The output of the system is usable by a clinician for determining a treatment for a new patient with solid and blood cancer.

According to other aspects of the present disclosure, the system may include one or more of the following features. The system may use a tumor-specific causal inference (TCI) algorithm applied to genomic data and transcriptomic data to discover driver SGAs and their target DEGs in various solid and blook cancers. The system may use different pattern discovery algorithm, e.g., various clustering algorithms, to extract the set of metagenes by using a clustering analysis to identify a reduced set of metagenes that exhibit co-expression patterns and drug response signals. A metagenes may comprise of tens to hundreds of genes exhibit co-expression pattern and share causal relationships with driver SGAs. The expression values of a metagene in a tumor may be computed from single-cell RNA, spatial transcriptome, or bulk RNA sample of a tumor profiled using microarray or RNA sequencing, by applying a gene set variation analysis or calculating the mean expression values of member genes. The machine learning model may comprise a classifier selected from the group consisting of a logistic regression model, a probit regression model, a linear discriminant analysis model, a Naïve Bayes model, a support vector machines model, a decision tree and random forest model, a neural networks model, and Lasso and Ridge logistic regression model.

According to another aspect of the present disclosure, a computer-implemented method for training and validating a machine learning model to predict efficacy of chemotherapy and biological agents in cancer treatment is provided. The method comprises collecting genomic and transcriptomic data of a cancer of interest. The method comprises applying the collected genes and related gene expression data to a tumor-specific causal inference module to discover cancer driver genes and their target differentially expressed genes. The method comprises analyzing patterns of the differentially expressed genes and identifying metagenes. The method comprises collecting genomic and gene expression data of cancer patients with known treatments and clinical outcomes. The method comprises training multiple classifiers. The method comprises predicting response to the treatments. The method comprises validating models using data from randomized controlled trials or observational data.

According to other aspects of the present disclosure, the method may include one or more of the following features. The method may further comprise, after training the machine learning model, collecting tumor tissue samples of a new patient diagnosed with the cancer. The method may further comprise profiling transcriptome of the samples. The method may further comprise mapping the transcriptome to the set of metagenes. The method may further comprise classifying, with the model, the efficacy of the biological agent for the new patient based on the mapping of the transcriptome for the new patient to the set of metagenes. The solid or blood cancer may comprise breast cancer, prostate cancer, colorectal cancer, esophageal cancer, stomach cancer, pancreatic cancer, liver cancer, kidney cancer, bladder cancer, skin cancer, uterine cancer, cervical cancer, ovarian cancer, head and neck cancer, thyroid cancer, central nervous system tumors, bone and soft tissue tumors, adrenocortical carcinoma, neuroendocrine tumors, mesothelioma, chordoma, Merkel cell carcinoma, leukemia, lymphoma, myeloma, and other hematologic malignancies.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is a flow chart illustrates the DeepRx system.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The DeepRx system may comprise a machine learning-assisted platform configured to support the selection of drugs for forming treatment regimens in patients with solid and blood cancers. The system may be designed to analyze various cancer types, including colorectal cancer, breast cancer, prostate cancer, and gastrointestinal cancers, among others. The DeepRx system may employ computational methods to process genomic and transcriptomic data for treatment prediction purposes.

The machine learning approach of the DeepRx system may utilize causal discovery methods to identify cancer driver SGAs and their associated target DEGs that may be involved in disease development processes. The system may analyze co-expression patterns among the target DEGs to construct a set of metagenes, where the expression values of the metagenes may reflect states of cellular signaling systems within tumor samples.

In some cases, the DeepRx system may use the metagenes as features to represent tumor characteristics for machine learning analysis. The system may include classification models that can be trained to predict whether tumor cells may be sensitive to specific biological and chemical agents. The classification approach may enable the system to provide predictions regarding treatment efficacy for individual patients.

The DeepRx system may comprise multiple components working in coordination to process cancer-related data and generate treatment predictions. A tumor-specific causal inference (TCI) model may be included for identifying cancer driver genes and their target DEGs. The system may also include a module for metagenes discovery that processes the identified DEGs to extract relevant co-expression patterns. Additionally, the system may incorporate one or more classifiers that can be trained with patient data and validated to predict patient responses to various biological and chemical treatment agents.

The machine learning framework of the DeepRx system may be configured to process large datasets of genomic and transcriptomic information from cancer patients. The system may analyze this data to identify patterns and relationships that may be predictive of treatment outcomes. The computational approach may enable the system to provide clinicians with data-driven insights for treatment selection in cancer patients.

The DeepRx system may be implemented as a computer-implemented architecture that leverages computational resources to perform machine learning analysis for cancer treatment prediction. A computer system may comprise one or more processors configured to execute instructions for processing genomic and transcriptomic data. The processors may access and execute software instructions that are stored in computer memory to carry out the various computational tasks associated with the DeepRx system.

The computer memory may store program instructions that define the operational logic of the DeepRx system components. The memory may contain executable code for implementing the tumor-specific causal inference model, the metagenes discovery module, and the classification algorithms. The processors may retrieve these instructions from the computer memory and execute them to perform data analysis operations on cancer-related datasets.

In some cases, the computer system may utilize multiple processors working in parallel to handle the computational demands of processing large genomic datasets. The processors may be configured to execute instructions that implement machine learning algorithms for identifying cancer driver genes and analyzing gene expression patterns. The distributed processing approach may enable the system to handle complex computational tasks associated with causal discovery methods and classification model training.

The computer memory may store both the executable instructions and the data structures used by the DeepRx system during operation. The memory may contain genomic data, transcriptomic data, and intermediate computational results generated during the analysis process. The processors may access this stored data through memory operations while executing the analysis algorithms.

The computer-implemented architecture may support the execution of multiple computational modules simultaneously. The processors may execute instructions for the TCI model to multiple tumor samples in parallel. Discovery of metagenes can utilize parallel computing to speed up the process of clustering. The memory management system may coordinate data access and storage operations to support the parallel execution of these different computational components.

The system architecture may be designed to accommodate the storage and processing requirements associated with large-scale genomic analysis. The computer memory may be configured to store extensive datasets containing gene expression information from multiple cancer patients. The processors may execute instructions that implement data preprocessing, feature extraction, and machine learning model training procedures on these stored datasets.

The tumor-specific causal inference (TCI) module may serve as a computational component within the DeepRx system for discovering cancer driver genes and their associated target differentially expressed genes (DEGs). The TCI module may implement algorithms designed to analyze relationships between genetic variations and gene expression changes in cancer samples. The module may process input data to identify causal relationships that may contribute to cancer development and progression.

The TCI module may be configured to accept dual data inputs comprising both genomic data and transcriptomic data from cancer samples. The genomic data may include information about genetic variations, mutations, copy number alterations, and structural variants present in tumor samples. In some cases, the genomic data may encompass single nucleotide polymorphisms, insertions, deletions, and chromosomal rearrangements that may be associated with cancer phenotypes.

The transcriptomic data processed by the TCI module may comprise gene expression measurements obtained from tumor samples through various sequencing technologies. The transcriptomic data may include RNA sequencing data that quantifies the expression levels of genes across different samples. In some cases, the transcriptomic data may contain normalized expression values, raw read counts, or other quantitative measures of gene activity in cancer cells.

The tumor-specific causal inference algorithm implemented within the TCI module may analyze the dual data inputs to establish causal relationships between genomic alterations and changes in gene expression. The algorithm may apply statistical methods to distinguish between correlation and causation in the relationships observed between genetic variations and expression patterns. The causal inference approach may enable the identification of driver genes that may directly influence the expression of downstream target genes.

The processing workflow of the TCI module may involve multiple computational steps for analyzing the genomic and transcriptomic data inputs. The module may first preprocess the raw data to remove noise, normalize expression values, and filter out low-quality measurements. The preprocessing steps may prepare the data for subsequent causal analysis by ensuring consistency and reliability of the input datasets.

During the causal discovery process, the TCI module may apply algorithms that evaluate potential causal relationships between genetic alterations and gene expression changes. The module may use statistical tests and computational models to assess the strength and direction of causal influences between different genetic elements. The causal inference algorithm may generate scores or probabilities that quantify the likelihood of causal relationships between driver genes and their potential targets.

The TCI module may identify cancer driver genes through analysis of the causal network structures derived from the dual data inputs. The module may distinguish driver genes from passenger genes by evaluating their causal influence on downstream gene expression patterns. Driver genes identified by the module may represent genetic elements that may directly contribute to cancer development through their effects on cellular pathways and biological processes.

The target differentially expressed genes (DEGs) discovered by the TCI module may represent genes whose expression levels may be causally influenced by the identified driver genes. The module may analyze expression differences between cancer samples and normal controls to identify genes that may be dysregulated in response to driver gene alterations. The DEGs may encompass both upregulated and downregulated genes that may participate in cancer-related biological processes.

The output of the TCI module may comprise a curated set of cancer driver genes along with their associated target DEGs that may be relevant for subsequent analysis steps in the DeepRx system. The module may provide quantitative measures of the causal relationships, including confidence scores and statistical significance values for the identified gene pairs. The processed results may serve as input for downstream modules that may perform metagenes discovery and classification model training.

The metagenes discovery module may process the target differentially expressed genes (DEGs) identified by the TCI module to extract a reduced set of representative gene groups that may capture cellular signaling states relevant to drug response prediction. The metagenes discovery process may involve computational analysis of co-expression relationships among the target DEGs to identify groups of genes that may exhibit coordinated expression patterns across different cancer samples.

The clustering analysis implemented within the metagenes discovery module may evaluate expression correlations between different target DEGs to identify subsets of genes that may be co-expressed under various biological conditions. The clustering algorithm may group genes based on similarity measures calculated from their expression profiles and shared common driver SGAs across multiple cancer samples. The analysis may identify gene clusters where members may exhibit similar expression changes in response to cellular perturbations or treatment conditions.

The clustering analysis may be configured to identify metagenes that may exhibit clean co-expression patterns characterized by strong internal correlations within gene groups and distinct separation between different clusters. The analysis may evaluate the coherence of expression patterns within each potential metagene to ensure that constituent genes may demonstrate consistent co-expression behavior across different sample conditions. The clustering approach may filter out genes that may exhibit inconsistent or noisy expression patterns that could reduce the reliability of downstream predictions.

In some cases, the clustering analysis may also assess the strength of drug response signals associated with different gene clusters to identify metagenes that may be most relevant for treatment prediction purposes. The analysis may evaluate correlations between metagene expression patterns and known drug response outcomes to prioritize gene clusters that may demonstrate strong associations with treatment efficacy. The drug response signal assessment may help select metagenes that may provide informative features for classification model training.

The metagenes discovery process may generate a reduced set of metagenes. The reduction from the whole transcriptome to disease-driving SGAs and their target DEGs and further to this smaller number of metagenes may enable more efficient computational processing while preserving the biological information relevant for drug response prediction. In some cases, the reduced set may provide a balance between computational efficiency and biological representation.

The expression value computation for the identified metagenes may be performed using gene set variation analysis (GSVA) or single-sample gene set enrichment analysis (ssGSEA) methods. The GSVA method may calculate expression scores for each metagene by analyzing the collective expression behavior of the constituent genes within each metagene cluster. The GSVA approach may transform individual gene expression measurements into metagene-level scores that may represent the overall activity state of each gene cluster.

The GSVA computation process may evaluate the expression levels of genes within each metagene relative to the overall expression distribution observed across all genes in a sample. The method may generate enrichment scores that may quantify whether the genes in a particular metagene may be expressed at higher or lower levels compared to the background gene expression distribution. The GSVA scores may provide normalized measures of metagene activity that may be comparable across different samples and experimental conditions.

The computed expression values for the metagenes may serve as feature representations for individual tumor samples in subsequent classification analysis. Each sample may be characterized by a vector of metagene expression values that may capture the relevant biological states associated with drug response mechanisms. The metagene-based representation may reduce the dimensionality of the original gene expression data while preserving the biological information that may be predictive of treatment outcomes.

The DeepRx system may incorporate various types of machine learning classifiers that can be trained to predict drug efficacy and treatment response in cancer patients. The classifiers may utilize the metagene expression values as input features to generate predictions regarding the sensitivity of tumor cells to specific biological and chemical agents. Different classifier architectures may be selected based on the characteristics of the dataset and the specific prediction requirements of the clinical application.

A logistic regression model may serve as one type of classifier within the DeepRx system for binary classification tasks related to drug response prediction. The logistic regression classifier may model the probability of treatment response as a function of the metagene expression values using a logistic function. The model may estimate coefficients for each metagene feature that may quantify the contribution of individual metagenes to the overall prediction of treatment efficacy. The logistic regression approach may provide interpretable results where the magnitude and direction of coefficients may indicate the relative importance of different metagenes in determining drug response.

In some cases, a probit regression model may be implemented as an alternative classifier for binary drug response prediction. The probit regression model may use a cumulative standard normal distribution function to model the relationship between metagene expression values and treatment response probabilities. The probit model may be particularly suitable for applications where the underlying biological processes may follow normal distribution assumptions. The probit regression classifier may generate probability estimates for treatment response that may be comparable to those produced by logistic regression models.

A linear discriminant analysis model may be incorporated as another classifier option within the DeepRx system for distinguishing between different treatment response categories. The linear discriminant analysis classifier may identify linear combinations of metagene features that may maximize the separation between responsive and non-responsive patient groups. The model may assume that the metagene expression values may follow multivariate normal distributions within each response category. The linear discriminant analysis approach may be computationally efficient and may perform well when the assumption of normal distributions may be satisfied by the input data.

The DeepRx system may also implement a Naïve Bayes model as a probabilistic classifier for drug response prediction. The Naïve Bayes classifier may calculate the probability of treatment response based on the assumption that metagene features may be conditionally independent given the response category. The model may apply Bayes' theorem to combine prior probabilities with likelihood estimates derived from the metagene expression data. The Naïve Bayes approach may be robust to small sample sizes and may provide reliable predictions even when the independence assumption may be violated to some degree.

A support vector machines model may be utilized as a classifier that can handle both linear and non-linear relationships between metagene features and drug response outcomes. The support vector machines classifier may identify an optimal hyperplane that may separate responsive and non-responsive patients in the metagene feature space. The model may use kernel functions to transform the input features into higher-dimensional spaces where linear separation may be possible. The support vector machines approach may be effective for handling high-dimensional data and may provide good generalization performance on new patient samples.

Decision tree and random forest models may be implemented as ensemble-based classifiers within the DeepRx system. A decision tree classifier may create a hierarchical set of decision rules based on metagene expression thresholds that may classify patients into different response categories. The decision tree approach may provide interpretable results where the decision path may indicate which metagene features may be most informative for predicting treatment response. A random forest model may combine multiple decision trees to improve prediction accuracy and reduce overfitting. The random forest classifier may generate predictions by aggregating the outputs of multiple individual decision trees trained on different subsets of the training data.

Neural networks models may serve as deep-learning-based classifiers that can capture complex non-linear relationships between metagene expression patterns and drug response outcomes. The neural networks classifier may comprise multiple layers of interconnected nodes that may transform the input metagene features through a series of weighted linear combinations and non-linear activation functions. The model may learn hierarchical representations of the input data that may capture both simple and complex patterns associated with treatment response. The neural networks approach may be particularly suitable for large datasets where complex feature interactions may be present.

Lasso and Ridge logistic regression models may be implemented as regularized versions of logistic regression that may address overfitting issues in high-dimensional datasets. The Lasso logistic regression model may apply L1 regularization to encourage sparsity in the model coefficients, potentially identifying a subset of metagenes that may be most relevant for drug response prediction. The Ridge logistic regression model may use L2 regularization to prevent large coefficient values and may improve the stability of predictions when multicollinearity may be present among metagene features. Both regularized approaches may enhance the generalization performance of the classifier on new patient data.

The selection of specific classifier types within the DeepRx system may depend on various factors including the size of the training dataset, the dimensionality of the metagene feature space, and the desired balance between prediction accuracy and model interpretability. Multiple classifiers may be trained and evaluated to identify the approach that may provide the best performance for specific cancer types and treatment scenarios. The trained classifiers may be validated using independent datasets to assess their ability to generalize to new patient populations.

The training process for the classifiers within the DeepRx system may utilize gene expression data collected from cancer patients with documented treatment histories and known clinical outcomes. The training dataset may comprise molecular profiling data from patients who have received specific biological and chemical agents, along with corresponding response measurements that may indicate treatment efficacy. The clinical outcome data may include various response metrics such as progression-free survival, overall survival, tumor response rates, and other measures of treatment effectiveness.

The transcriptomic data used for classifier training may encompass various types of molecular alterations present in patient tumor samples. The gene expression data may comprise transcriptomic measurements obtained from tumor samples through RNA sequencing or other expression profiling technologies. The expression data may provide quantitative measurements of gene activity levels that may be processed through the metagenes discovery pipeline to generate feature representations for classifier training.

The training process may involve preprocessing steps to prepare the expression data for machine learning analysis. The preprocessing procedures may include data normalization, quality control filtering, and feature selection steps that may enhance the reliability of the training dataset. Then, metagene expression values are calculated from transcriptomic data. The metagene expression values computed from the patient samples may serve as input features for the classifier training algorithms. The clinical outcome labels may be processed to create binary or categorical response variables that may represent different levels of treatment efficacy.

The classifier training algorithms may utilize supervised learning approaches that may learn relationships between metagene expression patterns and treatment response outcomes. The training process may involve optimization procedures that may adjust model parameters to minimize prediction errors on the training dataset. Cross-validation techniques may be applied during training to assess model performance and prevent overfitting to the training data. The training process may generate multiple candidate models that may be evaluated and compared to select the classifier configuration that may provide the best predictive performance.

The validation methodology for the trained DeepRx models may incorporate data from randomized controlled trials (RCTs) to assess the accuracy and reliability of treatment response predictions. RCT validation may provide a rigorous assessment of model performance using data from controlled clinical studies where patients may have been randomly assigned to different treatment groups. The RCT data may include genomic and transcriptomic profiling information from trial participants along with standardized outcome measurements collected according to clinical trial protocols.

The RCT validation process may evaluate the ability of trained classifiers to predict treatment responses in patient populations that may be independent from the training dataset. The validation analysis may compare model predictions with actual clinical outcomes observed in the randomized trials to calculate performance metrics such as sensitivity, specificity, positive predictive value, and negative predictive value. The RCT validation may assess whether the trained models may maintain their predictive accuracy when applied to new patient cohorts with different demographic and clinical characteristics.

In some cases, observational data may be utilized as an alternative or complementary approach for model validation. Observational validation datasets may comprise real-world clinical data collected from cancer patients who have received treatments outside of controlled trial settings. The observational data may include electronic health records, tumor registries, and other clinical databases that may contain molecular profiling information along with treatment and outcome data. The observational validation approach may assess model performance in more diverse patient populations that may reflect real-world clinical practice conditions.

The validation process using observational data may account for potential confounding factors and selection biases that may be present in non-randomized clinical datasets. Statistical methods may be applied to adjust for differences in patient characteristics, treatment protocols, and outcome measurement approaches that may vary across different clinical settings. The observational validation may provide insights into model performance across broader patient populations and treatment scenarios that may not be represented in controlled clinical trials.

The combined validation approach using both RCT and observational data may provide comprehensive assessment of model reliability and generalizability. The validation results may inform decisions regarding the clinical deployment of trained DeepRx models and may identify patient populations or treatment scenarios where model predictions may be most reliable. The validation process may also reveal limitations or biases in model performance that may guide further refinements to the training methodology or feature selection approaches.

The DeepRx system may be configured to analyze and generate treatment predictions for a comprehensive range of solid and blood cancer types. The machine learning framework may be adapted to process genomic and transcriptomic data from various malignancies, enabling the system to provide drug efficacy predictions across diverse cancer categories. The tumor-specific causal inference algorithms and metagenes discovery processes may be applied to different cancer types to identify disease-specific driver genes and expression patterns that may be relevant for treatment selection.

Colorectal cancer may represent one category of solid tumors where the DeepRx system may be applied to predict responses to biological and chemical agents. The system may analyze genomic alterations and gene expression patterns specific to colorectal malignancies to identify metagenes that may be associated with treatment efficacy. The classification models may be trained using data from colorectal cancer patients to predict sensitivity to agents such as oxaliplatin and bevacizumab, among other therapeutic options used in colorectal cancer treatment protocols.

In one embodiment, a validation study using large-scale phase III clinical trial data has demonstrated that the DeepRx system applied to colorectal cancer (COLOXIS model) can predict patient response to individual drugs (oxaliplatin and bevacizumab) as well as a combination regimen, such as FOLFOX, which is a chemotherapy regimen made up of the drugs folinic acid (leucovorin, FOL), fluorouracil (5-FU, F), and oxaliplatin (Eloxatin, OX).

Accurately predicting the efficacy of these drugs facilitates decision-making by clinicians and colorectal cancer (CRC) patients to determine whether a CRC patient should be treated with oxaliplatin. Accurate decisions will increase treatment efficacy as well as prevent overtreatment with oxaliplatin that only causes adverse effects.

The COLOXIS model is also predictive of the benefit of FOLFOX+Bev in the CRC adjuvant setting. This can enable a clinician to include bevacizumab in adjuvant therapy for a subset of patients to increase the treatment efficacy. The COLOXIS model can also be used to predict sensitivity to the above regimens (FOLFOX) or single drug (oxaliplatin or bevacizumab) in gastrointestinal (GI) cancers, such as esophageal cancer, gastric cancer, and colorectal cancer, in which the regimen and drugs are commonly used. This will enable clinicians to form individualized regimens by selecting effective drugs to treat GI cancer patients.

Breast cancer may constitute another solid tumor type where the DeepRx system may provide treatment prediction capabilities. The system may process molecular profiling data from breast cancer samples to identify cancer driver genes and target differentially expressed genes that may be characteristic of breast malignancies. The metagenes discovery module may extract co-expression patterns that may be specific to breast cancer biology and may be predictive of responses to hormonal therapies, chemotherapy agents, targeted biological treatments, and immunotherapy.

In one of the embodiments, the solid and blood cancer is breast cancer. The biological agent can comprise, for example, Adriamycin/cyclophosphamide-taxane (AC-T) and Taxane/cyclophosphamide (TC). In various implementations, GSVA scores for the set of metagenes for a cohort of patients are feature vectors for training the model through machine learning. In such cases, each patient in the cohort can be labeled as a responder or non-responder with respect to the biological agent. In various implementations, GSVA scores for the set of metagenes for a cohort of patients with a specific subtype breast cancer have one or more predetermined empirical feature vectors for training the model. In other implementations, GSVA scores for the set of metagenes for a cohort of patients have one or more empirical feature vectors for breast cancer metastasis status. In other implementations, GSVA scores for the set of metagenes for a cohort of patients have one or more empirical feature vectors for DeepRx Breast Cancer (DeepRx-BC).

In one of the embodiments, the DeepRX system can be trained and validated with large-scale phase III clinical trial data for breast cancer (DeepRx-BC model) to predict patient response to combination regimen Adriamycin/cyclophosphamide-taxane (AC-T) and Taxane/cyclophosphamide (TC) as well as combination regimen taxane/Carboplatin-Adriamycin/cyclophosphamide (TCab-AC) and individual drugs, such as capecitabin (Xerloda), gemcitabine, Bevcizumab (Avastin).

In another embodiment, the DeepRx-BC is developed to predict the pathologic complete response (pCR) in HER2-positive breast cancer. The DeepRx-BC can be trained to determine which HER2-positive patients (particularly HR+/HER2+ patients) will achieve pCR after neoadjuvant Taxane/carboplatin/Trastuzumab/Pertuzumab (TCHP) therapy. The DeepRx-BC can be trained to predict efficacy of anti-HER2 antibody-drug conjugate (ADC), such as trastuzumab deruxtecan (Enhertu, TDXd), ado-trastuzumab emtansine (Kadcyla, TDM1), disitamab vedotin (RC48), and SHR-A1811; as well as Trop-2 ADCs, such as Sacituzumab Govitecan (Trodelvy) and Datopotamab deruxtecan (Dato-DXd).

The DeepRX-BC model is also predictive of the benefit of anthracycline-containing regimen, such as Adriamycin/cyclophosphamide-taxane (AC-T) vs. non-anthracycline-containing regimen, such as Taxane-cyclophosphamide (TC), in the breast cancer adjuvant setting. This can enable a clinician to include anthracycline-containing regimen in adjuvant therapy for a subset of patients to increase the treatment efficacy. The DeepRx-BC model can also be used to predict sensitivity to the above regimens Adriamycin/cyclophosphamide-taxane (AC-T), Taxane-cyclophosphamide (TC), taxane/Carboplatin-Adriamycin/cyclophosphamide (TCab-AC) or single drug, capecitabin (Xerloda), gemcitabine, Bevcizumab (Avastin), immune checkpoint inhibitors (ICI) in various subtype of breast cancers, such as triple-negative breast cancer (TNBC) in which the regimen and drugs are commonly used. This will enable clinicians to form individualized regimens by selecting effective drugs to treat breast cancer patients.

In another embodiment, the DeepRx-BC is developed to predict benefit and tolerability of endocrine therapies, such as tamoxifen, anastrozole, letrozole, exemestane, and fulvestrant in the metastatic and adjuvant settings.

Prostate cancer may represent an additional solid tumor category where the DeepRx system may be utilized for drug efficacy prediction. The system may analyze prostate-specific genomic and transcriptomic data to identify molecular patterns that may be associated with treatment responses in prostate malignancies. The classification algorithms may be trained to predict responses to androgen deprivation therapies, chemotherapy regimens, and other treatment modalities commonly used in prostate cancer management.

In one of the embodiments, the DeepRx system can be trained and validated with large-scale phase III clinical trial data for prostate cancer (DeepRx-PS model) to predict patient response to both docetaxel and novel hormonal agents, such as abiraterone, enzalutamide, or apalutamide, response in prostate cancer.

The DeepRX-PS model is also predictive of the benefit of both docetaxel and novel hormonal agents, such as abiraterone, enzalutamide, or apalutamide, in the prostate cancer adjuvant setting. This can enable a clinician to include docetaxel or novel hormonal agents, such as abiraterone, enzalutamide, or apalutamide, or both in adjuvant therapy for a subset of patients to increase the treatment efficacy. This will enable clinicians to form individualized regimens by selecting effective drugs to treat prostate cancer patients.

Gastrointestinal cancers may encompass a broader category of malignancies where the DeepRx system may be applied across multiple anatomical sites within the digestive system. The system may process data from various gastrointestinal tumor types to identify common and site-specific molecular patterns that may be predictive of treatment outcomes. The machine learning models may be configured to account for the heterogeneity present across different gastrointestinal cancer subtypes while identifying shared biological pathways that may be relevant for drug response prediction.

Digestive system malignancies may include esophageal cancer and stomach cancer where the DeepRx system may provide drug efficacy predictions. Esophageal cancer may be analyzed to identify molecular patterns that may be predictive of responses to chemotherapy combinations, targeted therapies, and immunotherapy agents used in esophageal cancer treatment. Stomach cancer may be processed through the system to predict responses to chemotherapy regimens, anti-HER2 therapies, and other treatment modalities used in gastric cancer management.

Pancreatic cancer may represent a specific gastrointestinal malignancy where the DeepRx system may provide treatment prediction capabilities. The system may analyze the complex molecular landscape of pancreatic tumors to identify driver genes and expression patterns that may be associated with responses to chemotherapy combinations and targeted therapies. The challenging treatment landscape of pancreatic cancer may benefit from the data-driven approach provided by the DeepRx system for identifying patients who may respond to available therapeutic options.

Liver cancer may constitute another solid tumor type where the DeepRx system may be utilized for treatment prediction. The system may analyze hepatocellular carcinoma and other liver malignancies to identify molecular patterns that may be associated with responses to targeted therapies, immunotherapy agents, anti-angiogenesis agents, and locoregional treatments. The complex molecular landscape of liver cancer may be addressed through the causal inference algorithms that may identify driver genes specific to hepatic malignancies.

Kidney cancer may constitute another solid tumor type where the DeepRx system may be applied to predict treatment efficacy. The system may process renal cell carcinoma data to identify molecular patterns that may be predictive of responses to immunotherapy agents, targeted kinase inhibitors, and other treatment modalities used in kidney cancer management. The classification models may be trained to distinguish between patients who may benefit from different therapeutic approaches based on their tumor molecular profiles.

Bladder cancer may represent an additional urological malignancy where the DeepRx system may provide drug response predictions. The system may analyze bladder tumor samples to identify genomic alterations and expression patterns that may be associated with responses to chemotherapy regimens, targeted treatments, immunotherapy agents, and antibody-drug-conjugates (ADCs). The molecular heterogeneity of bladder cancer may be addressed through the causal inference algorithms that may identify subtype-specific driver genes and therapeutic targets.

Skin cancer may encompass various malignancies including melanoma and non-melanoma skin cancers where the DeepRx system may be applied. The system may process molecular data from skin cancer samples to identify patterns that may be predictive of responses to immunotherapy agents, and targeted therapies. The classification models may be particularly relevant for melanoma treatment selection where multiple therapeutic options may be available.

Gynecological malignancies may represent another category where the DeepRx system may provide treatment prediction capabilities. Uterine cancer may be analyzed through the system to identify molecular patterns that may be associated with responses to hormonal therapies, chemotherapy regimens, targeted treatments, and antibody-drug-conjugates (ADCs). Cervical cancer may be processed through the DeepRx algorithms to predict responses to chemotherapy combinations and radiation sensitizers used in cervical cancer treatment protocols. Ovarian cancer may benefit from the system's ability to analyze complex molecular patterns that may be predictive of responses to platinum-based chemotherapy, PARP inhibitors, other targeted therapies, and antibody-drug-conjugates (ADCs) commonly used in ovarian cancer management.

Head and neck cancer may represent another solid tumor category where the DeepRx system may be applied to predict treatment responses. The system may analyze molecular data from head and neck squamous cell carcinomas to identify patterns that may be associated with responses to chemotherapy, targeted therapies, immunotherapy agents, and antibody-drug-conjugates (ADCs). The molecular heterogeneity of head and neck cancers across different anatomical subsites may be addressed through the tumor-specific causal inference algorithms.

Thyroid cancer may constitute an endocrine malignancy where the DeepRx system may provide treatment prediction capabilities. The system may process thyroid cancer data to identify molecular patterns that may be predictive of responses to radioactive iodine therapy, targeted kinase inhibitors, and other treatment modalities used in thyroid cancer management. The different histological subtypes of thyroid cancer may be analyzed through the system to identify subtype-specific therapeutic targets.

Hematologic malignancies may represent a distinct category of blood cancers where the DeepRx system may be applied. Leukemia may encompass various subtypes including acute and chronic forms where the system may predict responses to chemotherapy regimens, targeted therapies, and immunotherapy approaches. Lymphoma may include Hodgkin and non-Hodgkin subtypes where the DeepRx system may analyze molecular patterns that may be predictive of responses to chemotherapy combinations, immunotherapy agents, and targeted treatments. Myeloma may represent another hematologic malignancy where the system may predict responses to proteasome inhibitors, immunomodulatory drugs, and other targeted therapies used in multiple myeloma treatment.

Central nervous system tumors may constitute a specialized category of malignancies where the DeepRx system may provide treatment prediction capabilities. The system may analyze brain tumor samples to identify molecular patterns that may be associated with responses to chemotherapy agents, targeted therapies, and radiation sensitizers. The unique challenges associated with drug delivery across the blood-brain barrier may be addressed through the identification of molecular biomarkers that may predict treatment efficacy in central nervous system malignancies.

Bone and soft tissue tumors may represent another category of sarcomas where the DeepRx system may be applied. The system may process molecular data from various sarcoma subtypes to identify patterns that may be predictive of responses to chemotherapy regimens, targeted therapies, and other treatment modalities used in sarcoma management. The molecular diversity of sarcoma subtypes may be addressed through the causal inference algorithms that may identify subtype-specific driver genes and therapeutic targets.

Rare solid tumors may encompass various uncommon malignancies where the DeepRx system may provide treatment prediction capabilities despite limited patient populations. Adrenocortical carcinoma may be analyzed through the system to identify molecular patterns that may be associated with responses to chemotherapy agents and targeted therapies used in this rare endocrine malignancy. Neuroendocrine tumors may be processed through the DeepRx algorithms to predict responses to somatostatin analogs, targeted therapies, and chemotherapy regimens used in neuroendocrine tumor management.

Mesothelioma may represent another rare solid tumor where the DeepRx system may be applied to predict treatment responses. The system may analyze mesothelioma samples to identify molecular patterns that may be predictive of responses to chemotherapy combinations, immunotherapy agents, and targeted treatments. Chordoma may constitute a rare bone tumor where the system may provide treatment predictions based on molecular profiling data. Merkel cell carcinoma may represent a rare skin cancer where the DeepRx system may predict responses to immunotherapy agents and other treatment modalities used in this uncommon malignancy.

The DeepRx system may be configured to generate predictions regarding the efficacy of specific biological and chemical agents used in cancer treatment protocols. The classification models within the system may be trained to predict patient responses to individual therapeutic compounds by analyzing the relationships between metagene expression patterns and known drug response outcomes. The drug-specific prediction capability may enable clinicians to select treatments that may be most likely to provide therapeutic benefit for individual patients based on their tumor molecular profiles.

The prediction process for specific drug efficacy may utilize the metagene expression values as input features to generate probability estimates or classification scores that may indicate the likelihood of treatment response. The trained classifiers may process the metagene-based tumor representation to identify molecular patterns that may be associated with sensitivity or resistance to particular therapeutic agents. The drug-specific models may be developed through training on datasets that may include patients who have received specific treatments along with corresponding response measurements.

In colorectal cancer applications, the DeepRx system may be configured to predict the efficacy of oxaliplatin, a platinum-based chemotherapy agent commonly used in colorectal cancer treatment regimens. The oxaliplatin prediction model may analyze metagene expression patterns that may be associated with DNA repair mechanisms, cell cycle regulation, and apoptotic pathways that may influence cellular responses to platinum-based DNA damage. The classifier may identify colorectal cancer patients whose tumors may exhibit molecular characteristics that may be predictive of sensitivity to oxaliplatin treatment.

The oxaliplatin efficacy prediction may involve analysis of metagenes that may represent genes involved in nucleotide excision repair, mismatch repair, and homologous recombination pathways. The expression patterns of these DNA repair-related metagenes may provide information about the tumor's capacity to respond to platinum-induced DNA damage. Tumors with deficient DNA repair mechanisms may be more likely to respond to oxaliplatin treatment, while tumors with enhanced repair capabilities may exhibit resistance to platinum-based therapy.

The DeepRx system may also be configured to predict the efficacy of bevacizumab, an anti-angiogenic monoclonal antibody that may be used in combination with chemotherapy for colorectal cancer treatment. The bevacizumab prediction model may analyze metagene expression patterns that may be associated with angiogenesis, vascular development, and hypoxia response pathways that may influence tumor responses to anti-angiogenic therapy. The classifier may identify patients whose tumors may exhibit molecular characteristics that may be predictive of sensitivity to bevacizumab treatment.

The bevacizumab efficacy prediction may focus on metagenes that may represent genes involved in vascular endothelial growth factor signaling, endothelial cell proliferation, and blood vessel formation processes. The expression levels of angiogenesis-related metagenes may provide information about the tumor's dependence on neovascularization for growth and survival. Tumors with high angiogenic activity may be more likely to respond to bevacizumab treatment, while tumors with low angiogenic dependence may exhibit limited responses to anti-angiogenic therapy.

The combination prediction capability of the DeepRx system may enable simultaneous assessment of both oxaliplatin and bevacizumab efficacy for colorectal cancer patients. The system may generate separate prediction scores for each agent that may be combined to assess the potential effectiveness of combination therapy regimens. The dual prediction approach may help identify patients who may benefit from both agents, patients who may respond to one agent but not the other, or patients who may be unlikely to respond to either treatment.

The drug-specific prediction models may be trained using clinical datasets that may include colorectal cancer patients who have received oxaliplatin-based chemotherapy regimens, bevacizumab-containing treatment protocols, or combination therapies that may include both agents. The training data may comprise molecular profiling information from patient tumor samples along with corresponding clinical outcomes such as response rates, progression-free survival, and overall survival measurements. The supervised learning algorithms may identify metagene expression patterns that may be most predictive of responses to each specific agent.

The validation of drug-specific efficacy predictions may involve assessment of model performance using independent datasets from colorectal cancer patients who have received the targeted therapies. The validation process may evaluate the accuracy of oxaliplatin response predictions by comparing model outputs with actual clinical responses observed in patients treated with oxaliplatin-containing regimens. Similarly, bevacizumab prediction accuracy may be assessed through comparison with clinical outcomes from patients who have received bevacizumab-based treatments.

The clinical implementation of drug-specific efficacy predictions may involve processing of tumor samples from newly diagnosed colorectal cancer patients to generate treatment recommendations. The tumor tissue may be subjected to transcriptomic profiling to obtain gene expression measurements that may be processed through the metagenes discovery pipeline. The resulting metagene expression values may be input to the trained oxaliplatin and bevacizumab prediction models to generate efficacy scores that may inform treatment selection decisions.

The drug-specific prediction capability may extend beyond oxaliplatin and bevacizumab to encompass other therapeutic agents commonly used in colorectal cancer treatment. The system may be configured to predict responses to fluoropyrimidine agents, irinotecan, cetuximab, panitumumab, and other targeted therapies through similar metagene-based classification approaches. Each drug-specific model may focus on metagenes that may represent biological pathways and molecular mechanisms that may be relevant to the particular agent's mechanism of action.

The mechanistic basis for drug-specific predictions may involve analysis of metagenes that may represent cellular processes that may be directly targeted by specific therapeutic agents. For cytotoxic chemotherapy agents, the relevant metagenes may include those associated with cell cycle regulation, DNA damage response, and apoptotic signaling pathways. For targeted biological agents, the relevant metagenes may focus on specific signaling pathways, receptor expression levels, and downstream effector mechanisms that may be modulated by the therapeutic intervention.

The integrated workflow of the DeepRx system may coordinate multiple computational modules to process new patient samples and generate treatment predictions through a systematic analytical pipeline. The complete system integration may involve sequential processing steps that may transform raw tumor tissue samples into actionable treatment recommendations through coordinated operation of the TCI module, metagenes discovery components, and trained classification algorithms.

The patient sample processing workflow may begin with the collection of tumor tissue samples from new patients who have been diagnosed with cancer and may require treatment selection guidance. The tumor tissue collection process may involve obtaining fresh or frozen tissue specimens through biopsy procedures, surgical resection, or other clinical sampling methods. The collected tissue samples may be processed to extract high-quality RNA that may be suitable for transcriptomic profiling analysis. The tissue processing procedures may include quality control steps to ensure that the extracted RNA may meet the requirements for downstream sequencing and expression analysis.

The transcriptome profiling of the collected tumor samples may be performed using RNA sequencing technologies or other gene expression measurement platforms. The transcriptomic profiling process may generate comprehensive gene expression data that may quantify the activity levels of thousands of genes present in the tumor tissue. The sequencing procedures may produce raw expression data that may require preprocessing steps including read alignment, expression quantification, and normalization to generate standardized gene expression measurements that may be compatible with the DeepRx analytical pipeline.

The transcriptome mapping process may involve computational procedures that may transform the comprehensive gene expression data from new patient samples into the metagene-based representation used by the trained classification models. The mapping workflow may utilize the predefined set of metagenes that may have been identified during the system training phase through the TCI module and metagenes discovery processes. The transcriptome-to-metagene mapping may ensure that new patient samples may be represented using the same feature space that may have been used for training the predictive models.

The gene set variation analysis (GSVA) computation may be applied during the transcriptome mapping process to calculate expression values for each metagene based on the expression levels of constituent genes in the new patient sample. The GSVA algorithm may evaluate the collective expression behavior of genes within each metagene cluster to generate normalized expression scores that may represent the activity state of each metagene in the patient's tumor. The computed metagene expression values may serve as input features for the trained classification algorithms.

The system integration workflow may coordinate the processing of new patient data through the established analytical framework that may have been developed during the training phase. The TCI module results may define the cancer driver genes and target differentially expressed genes that may be relevant for the specific cancer type being analyzed. The metagenes discovery module output may specify the gene clusters and co-expression patterns that may be used for feature representation. The trained classifiers may utilize these predefined molecular features to generate treatment predictions for the new patient samples.

The classification process for new patients may involve applying the trained machine learning models to the metagene expression values computed from the patient's tumor transcriptome. The classification algorithms may process the metagene-based feature representation to generate prediction scores or probability estimates that may indicate the likelihood of treatment response to specific biological and chemical agents. The classification output may provide quantitative measures of predicted treatment efficacy that may be interpreted by clinicians for treatment selection purposes.

The efficacy classification based on the transcriptome mapping may utilize the established relationships between metagene expression patterns and drug response outcomes that may have been learned during the model training phase. The trained classifiers may recognize molecular patterns in the new patient's metagene profile that may be similar to patterns observed in the training data from patients with known treatment outcomes. The pattern recognition capability may enable the system to extrapolate from historical patient data to generate predictions for new patients with similar molecular characteristics.

The integrated workflow may accommodate multiple drug-specific prediction models that may be applied simultaneously to generate comprehensive treatment recommendations for new patients. The system may process the same metagene expression profile through different trained classifiers that may be specific to various therapeutic agents. The multi-drug prediction capability may enable comparison of predicted efficacy across different treatment options to identify therapies that may be most likely to provide clinical benefit for the individual patient.

The workflow coordination may ensure consistency in data processing procedures between the training phase and the new patient analysis phase. The transcriptome profiling, preprocessing, and metagene computation steps applied to new patient samples may follow the same protocols and algorithms that may have been used during the system development and training phases. The standardized processing approach may ensure that new patient data may be compatible with the trained models and may enable reliable prediction performance.

The system integration may also incorporate quality control procedures that may assess the reliability of predictions generated for new patients. The quality control analysis may evaluate whether the new patient's molecular profile may fall within the range of characteristics represented in the training data. Samples with molecular profiles that may be significantly different from the training data distribution may be flagged for additional review or may receive modified confidence scores for their treatment predictions.

The complete workflow output may provide clinicians with treatment efficacy predictions along with associated confidence measures and supporting molecular information that may inform clinical decision-making. The prediction results may include drug-specific efficacy scores, probability estimates for treatment response, and identification of the molecular features that may contribute most strongly to the predictions. The comprehensive output may enable clinicians to evaluate the predicted treatment options in the context of other clinical factors and patient characteristics.

The integrated DeepRx system workflow may be designed to accommodate various cancer types through the application of cancer-specific TCI models, metagenes, and trained classifiers. The system may maintain separate analytical pipelines for different cancer types while utilizing the same overall workflow structure for processing new patient samples. The cancer-specific customization may ensure that the molecular features and prediction models may be appropriate for the particular biological characteristics and treatment landscape associated with each cancer type.

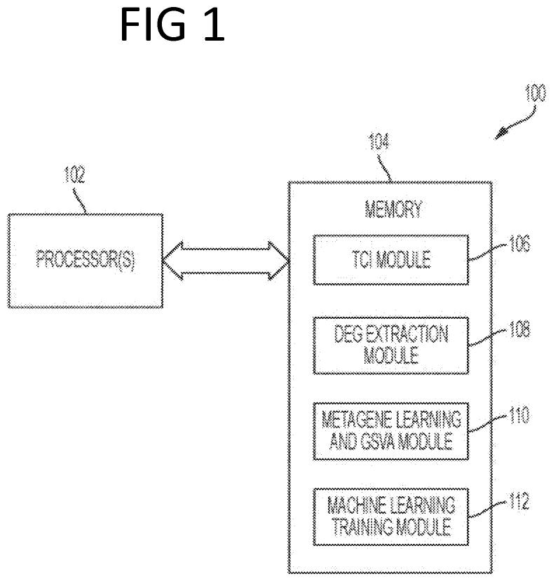

FIG. 1 is a diagram of a computer system 100 that could be used to implement the embodiments described above. The illustrated computer system 100 includes one or more processors 102 and one or more memory units 104 that are in communication via a data bus and/or electronic data network. For simplicity, only one processor 102 and one memory unit 104 are shown in FIG. 1. The memory 104 may store various software modules 106, 108, 110, and 112 that comprise software or computer instructions to be executed by the processor 102. the TCI module 106 may include software for performing the TCI analysis; the DEG extraction module 108 may include software for performing the target DEG extraction; the metagene learning and GSVA module 110 may include software for performing the metagene learning and GSVA.

The processor(s) 102 may include one or more CPU cores, GPU cores, and/or Al accelerator cores, or Virtual Processor cores. The memory 104 may comprise primary computer memory, such as a read-only memory (ROM) and/or a random access memory (e.g., RAM). The memory 104 could also comprise secondary memory, such as magnetic or optical disk drives or flash memory, for example. The software modules 106, 108, 110, and 112 may be implemented in computer software using any suitable computer programming language such as . NET, C, C++, or Python, and using conventional, functional, or object-oriented techniques.

Programming languages for computer software and other computer-implemented instructions may be translated into machine language by a compiler or an assembler before execution and/or may be translated directly at run time by an interpreter. Examples of assembly languages include ARM, MIPS, and x86; examples of high-level languages include Ada, BASIC, C, C++, C#, Python, R, COBOL, Fortran, Java, Lisp, Pascal, Object Pascal, Haskell, ML; and examples of scripting languages include Bourne shell script, JavaScript, Python, Ruby, Lua, PHP, and Perl. The various data used in the process of FIG. 1 may be stored in primary, secondary, tertiary, and/or offline (e.g., cloud) storage.

Computer system 100 can be of varying types including a workstation, server, computing cluster, blade server, server farm, or any other data processing system or computing device. The computer system 100 could be, for example, a server on a cloud network. Due to the ever-changing nature of computers and networks, the description of computer system 100 depicted in FIG. 1 is intended only as a specific example for the purposes of illustrating some implementations. Many other configurations of computer system 100 are possibly having more or fewer components than the computer system depicted in FIG. 1.

A number of implementations have been described. Nevertheless, it will be understood that various modifications may be made without departing from the spirit and scope of the disclosure. Accordingly, other implementations are within the scope of the following claims.

1. A computer-implemented system for training a machine learning model to predict efficacy of a biological agent for solid and blood cancers, comprising:
one or more processors that execute instructions stored in computer memory;
wherein the one or more processors are configured to:
identify target differentially expressed genes for a cancer using a tumor-specific causal inference algorithm applied to genomic data and transcriptomic data;
compute expression values for a set of metagenes from the target differentially expressed genes, wherein the set of metagenes exhibit co-expression patterns with respect to response to the biological agent for patients with the solid and blood cancer; and
train, through machine learning, the model to predict the efficacy of the biological agent for the cancer, such that an output of the model is usable by a clinician for determining a treatment for a new patient with the solid and blood cancer.

2. The computer-implemented system of claim 1, wherein the one or more processors are further configured to extract the set of metagenes by using a clustering analysis to identify a reduced set of metagenes that exhibit clean co-expression patterns and strong drug response signals.

3. The computer-implemented system of claim 2, wherein the reduced set of metagenes comprises ten to twenty metagenes.
The computer-implemented system of claim 2, wherein the reduced set of metagenes.

5. The computer-implemented system of claim 1, wherein the expression values are computed using a gene set variation analysis.

6. The computer-implemented system of claim 1, wherein the machine learning model comprises a classifier selected from the group consisting of a logistic regression model, a probit regression model, a linear discriminant analysis model, a Naïve Bayes model, a support vector machines model, a decision tree and random forest model, a neural networks model, and Lasso and Ridge logistic regression model.

7. The computer-implemented system of claim 1, wherein the solid and blood cancer comprises breast cancer, prostate cancer, colorectal cancer, esophageal cancer, stomach cancer, pancreatic cancer, liver cancer, kidney cancer, bladder cancer, skin cancer, uterine cancer, cervical cancer, ovarian cancer, head and neck cancer, thyroid cancer, leukemia, lymphoma, myeloma, and other hematologic malignancies.

8. The computer-implemented system of claim 7, wherein the solid cancer comprises central nervous system tumors, bone and soft tissue tumors, adrenocortical carcinoma, neuroendocrine tumors, mesothelioma, chordoma, and Merkel cell carcinoma.

9. The computer-implemented system of claim 1, wherein the biological agent comprises oxaliplatin and bevacizumab for colorectal cancer treatment.

10. A computer-implemented method for training and validating a machine learning model to predict efficacy of chemotherapy and biological agents in cancer treatment, comprising:
collecting genomic and transcriptomic data of a cancer of interest;
applying the collected genomic and transcriptomic data to a tumor-specific causal inference module to discover cancer driver genes and their target differentially expressed genes;
analyzing patterns of the differentially expressed genes and identifying metagenes;
collecting genomic and gene expression data of cancer patients with known treatments and clinical outcomes;
training multiple classifiers using the metagenes and the genomic and gene expression data;
predicting response to the treatments using the trained classifiers; and
validating the machine learning model using data from randomized controlled trials or observational data.

11. The computer-implemented method of claim 10, wherein the step of analyzing patterns of the differentially expressed genes comprises using a clustering analysis to identify a reduced set of metagenes that exhibit clean co-expression patterns and strong drug response signals.

12. The computer-implemented method of claim 11, wherein the reduced set of metagenes comprises ten to twenty metagenes.

13. The computer-implemented method of claim 11, wherein expression values for the metagenes are computed using a gene set variation analysis.

14. The computer-implemented method of claim 10, wherein the multiple classifiers comprise classifiers selected from the group consisting of a logistic regression model, a probit regression model, a linear discriminant analysis model, a Naïve Bayes model, a support vector machines model, a decision tree and random forest model, a neural networks model, and Lasso and Ridge logistic regression model.

15. The computer-implemented method of claim 10, further comprising:
collecting tumor tissue samples of a new patient diagnosed with the cancer;
profiling transcriptome of the samples;
mapping the transcriptome to the set of metagenes; and
classifying, with the machine learning model, the efficacy of the biological agent for the new patient based on the mapping of the transcriptome for the new patient to the set of metagenes.

16. The computer-implemented method of claim 15, wherein the cancer comprises colorectal cancer and the biological agent comprises oxaliplatin and bevacizumab.

17. A computer-implemented method for predicting treatment efficacy for a cancer patient, comprising:
collecting tumor tissue samples of a patient diagnosed with a cancer;
profiling transcriptome of the tumor tissue samples;
mapping the transcriptome to a set of metagenes, wherein the set of metagenes are derived from target differentially expressed genes identified using a tumor-specific causal inference algorithm; and
classifying, with a trained machine learning model, efficacy of a biological agent for the patient based on the mapping of the transcriptome to the set of metagenes, wherein the trained machine learning model was trained using expression values of the set of metagenes that exhibit co-expression patterns with respect to response to the biological agent.

18. The computer-implemented method of claim 17, wherein the set of metagenes comprises ten to twenty metagenes that exhibit clean co-expression patterns and strong drug response signals.

19. The computer-implemented method of claim 18, wherein expression values for the set of metagenes are computed using a gene set variation analysis.

20. The computer-implemented method of claim 17, wherein the cancer comprises colorectal cancer and the biological agent comprises oxaliplatin and bevacizumab.
